(19) United States
(12) Patent Application Publication
(10) Pub. No.: US 2007/0212402 A1
(43) Pub. Date: Sep. 13, 2007

(54) Patch

(76) Inventors: Qainton Van Rooven, Windhoek (NA); Salmon Johannes Vermaak, Windhoek (NA); Volker Reinhard Schillack, Pretoria (ZA); Johannes Cronie, Pretoria (ZA)

Correspondence Address: HAHN AND MOODLEY, LLP, P.O. BOX 52050, MINNEAPOLIS, MN, 55402, US

(21) Appl. No.: 10/581,827

(22) PCT Filed: Dec. 1, 2004
(86) PCT No.: PCT/IB04/52616

§ 371 Date: May 23, 2007

(30) Foreign Application Priority Data
Dec. 5, 2003 (ZA) 2003/9481

(51) Int. Cl. A61F 13/00

(52) U.S. Cl. 424/449

(57) ABSTRACT

A patch device adapted for use in the transdermal administration to a patient of a composition including, or consisting of, a selected irritating substance, preferably dimethylformamide or DMF, the patch consisting of a layered construct adapted to be adhered to the skin of a patient and defining a depot cavity for the composition to be administered between a proximal layer and a distal layer thereof, which proximal layer is adapted in use to be located in intimate contact with the skin of the patient and which distal layer is in use disposed on the outer side thereof, the distal layer being characterised in that it is substantially impervious to the composition to be administered, and the proximal layer being characterised in that it is partially permeable to that composition, so that in use the composition may be disposed in the cavity and permeate from there through the proximal layer to be absorbed through the skin into the body of the patient, the proximal layer being further characterised in that its permeability to the irritating substance or component of such composition is less than the permeability of the patients skin to such irritating substance.

### FIELD OF THE INVENTION

This invention relates to a skin patch or plaster suitable for use in the transdermal delivery to the human or animal body of pharmaceutically active substances or compositions containing such active substances.

### BACKGROUND TO THE INVENTION

It is well known in the field of drug delivery that certain pharmaceutically active substances may be delivered transdermally to a patient in need thereof. It is also known that many pharmaceutically active substances are not suitable as such for transdermal administration as they are not readily absorbed through the skin or cause irritation to the skin when applied directly to the skin. It is also known that the absorption of such substances through the skin of the patient may in some cases be enhanced by the user of various excipients used in the formulation of pharmaceutical preparations and in particular by the use of substances known as penetration enhancing agents. It is however further known that some of the known penetration enhancing agents and other excipients conventionally used in the formulation of pharmaceutical preparations cause irritation when applied to the skin.

Various solutions have been proposed to overcome the problem of skin irritation by compositions intended to be used for transdermal administration of pharmaceutical preparations. The recently granted U.S. Pat. No. 6,579,865 to Mak et al discloses one such attempt and refers to several others.

It is also known that dimethylformamide (DMF) is useful as a penetration enhancing agent. It is referred to in that context in several patents including U.S. Pat. No. 6,214,374 (Schmirler, et al). It has also been suggested in WO 97/22248US that DMF may be used as an active ingredient in combating HIV-AIDS. In WO 99/13885 it is more generally suggested that DMF may be used to affect the immune response of the body to a variety of ailments. There has however not been a suitably reliable transdermal administration device that can be used for the purpose of administering DMF at the low and sub-toxic dosages that are considered necessary to apply such proposed treatment.

One of the difficulties with applying DMF by means of conventional transdermal devices, is that it causes severe skin irritation in the form of pruritic erythema DMF is in addition also a solvent for a large number of synthetic materials, and permeates through a number of others, both of which factors limit the choice of materials which may be used in constructing a patch suitable for use in administering DMF or DMF containing compositions to a patient—human or animal.

### OBJECT OF THE INVENTION

It is an object of the present invention to provide a patch construction which is suitable to be used in general, as a means transdermally to administer pharmaceutically active substances and compositions which are irritating to the skin when applied by conventional means, and more specifically for the administration of dimethylformamide as such or of compositions containing dimethylformamide.

### BRIEF DESCRIPTION OF THE INVENTION

According to the invention there is provided a patch device adapted for use in the transdermal administration to a patient of a composition including, or consisting of, a selected irritating substance, the patch consisting of a layered construct adapted to be adhered to the skin of a patient and defining a depot cavity for the composition to be administered between a proximal layer and a distal layer thereof, which proximal layer is adapted in use to be located in intimate contact with the skin of the patient and which distal layer is in use disposed on the outer side thereof, the distal layer being characterized in that it is substantially impervious to the composition to be administered, and the proximal layer being characterized in that it is partially permeable to that composition, so that in use the composition may be disposed in the cavity and permeate from there through the proximal layer to be absorbed through the skin into the body of the patient, the proximal layer being further characterized in that its permeability to the irritating substance or component of such composition is less than the permeability of the patient's skin to such irritating substance.

The layers of the patch may be produced from any suitable material or combination of materials and the chemical composition of the proximal and distal layers may be the same or different. Their respective permeability and impervious characteristics may be achieved by virtue of their thickness of the respective layers, or by other forms of modification of the layers. The distal layer may thus be modified by the application thereto of a thin impervious layer to render a composite layer which is impervious to the components of the composition to be applied by means of the patch. The patch is preferably flexible to be capable of moving with the skin for the sake of comfort, and is hence preferably produced from an elastomeric material, which is most preferably a silicone material, but it is also feasible to make the patch according to the invention with a distal sheet composed of or incorporating a thin sheet of aluminium.

By providing a patch from which the irritant substance is released at a rate which is lower than the rate at which the skin absorbs such substance, it has been found that the extent of irritation is substantially reduced. This particularly the case where the irritant is DMF. Without wishing to be bound by theory it is believed that one of the reasons why DMF leads to erythema is that the water content of perspiration released by the skin under a patch reacts with DMF to form an irritant, possibly formic acid if allowed to remain on the skin in a quantity that is greater than the quantity which the skin is capable of absorbing. By the present invention it is sought to provide a means whereby such irritants as DMF will be released at a rate below the rate at which skin absorption of such substance will take place, thereby ensuring that no build-up of such substances occurs on the skin. It provides a solution to the problem in that it materially reduces, if not completely eliminates, the occurrence of erythema. The invention accordingly specifically provides for a patch suitable for use in the administration of DMF to a patient and characterized in that the proximal layer of the patch has a permeability to DMF such that DMF which is, in use, located in the cavity between the layers will be released through the proximal layer at a rate below the rate at which it is absorbed through the skin of the patient to which the patch is in use applied, thereby substantially preventing build-up of DMF in direct contact with the skin of the patient.

The patch may be adapted to be adhered to the skin of the patient by being provided with a peripheral edge zone of the proximal layer being provided with a pharmaceutically acceptable adhesive layer. The adhesive layer may be covered by means of a conventional peel-off cover sheet during storage.

The layers of the layered patch construct may be of the same or different chemical compositions. In one preferred form of the invention the layers may be of the same chemical composition, but may be of different thickness and/or be otherwise modified to provide for the requisite degree of imperviousness of the distal layer and permeability of the proximal layer. The distal layer may thus be a composite layer including two or more layers of the same or different materials.

The layers of a patch intended to be used for the administration of dimethylformamide as such, or of a pharmaceutical preparation containing dimethylformamide as a penetration enhancing agent, may be of the same chemical composition and may be in the form of vulcanizates of silicone. In a preferred application of this form of the invention the proximal and distal layers of the patch construct may both be produced from a silicone composition that is commercially available and marketed by Wacker-Chemie GmbH of Germany under the trade name Elastosil® which composition may during the fabrication of the layers be vulcanized by means of Curing Agent E, C1 or C6 which is also obtainable from Wacker-Chemie as is known in the trade of the production of silicone articles. Curing Agent E is constituted by a 50% paste of bis-(2,4-dichlorobenzoyl)-peroxide in silicone fluid, Curing Agent C1 is Dicumyl peroxide (98%) and Curing Agent C6 is a 45% paste of 2,5-bis-(t-butylperoxy)-2,5-dimethyl-hexane in silicone rubber. In the preferred embodiment of the invention the layers are produced from the Elastosil® R401/70 by press vulcanization or injection moulding and with the aid of Curing Agent C1 or C6.

The composition of the layer intended for use as the proximal layer in a patch for use in the administration of dimethylformamide (DMF) as such, or of a pharmaceutical preparation containing dimethylformamide as a penetration enhancing agent, is preferably produced to have a permeability to dimethylformamide of not more than 9 mg DMF/cm²/hour. Where Elastosil® R401/70 is used as the proximal layer this rate of permeability may be achieved when the layer has a thickness of 0.25 mm. The value of 9 mg DMF/cm²/hr is slightly below the typical absorption rate of DMF through a human skin. The patch may preferably be configured to have a contact surface area of the proximal layer of 64 cm².

The composition of the layer intended for use as the distal layer in a patch for use in the administration of dimethylformamide (DMF) as such, or of a pharmaceutical preparation containing dimethylformamide as a penetration enhancing agent, is preferably produced to be substantially impervious to DMF. Where Elastosil® R401/70 is used as the distal layer this may be achieved when the layer has a sufficient thickness but such thickness may be considered undesirable as it compromises the flexibility and overall appearance of the patch. It is thus preferable to provide a modified or composite distal layer produced by the application of a DMF impervious layer of high density polypropylene to the inner surface thereof that in use faces the proximal layer of the patch. In this application the Elastosil® layer may typically be 0.4 mm in thickness (which as such is permeable to DMF) and a 50 micron thick high density polypropylene film is applied to the inner surface thereof with the aid of a suitable adhesive. The adhesive may comprise the silicone adhesive marketed in the transfer tape form under the code BRD577B by PPI Adhesive Products of Waterford in Ireland.

The cavity defined between the proximal and distal layers of the patch construct is preferably in use filled with a solid filler material to serve as a carrier for the pharmaceutically active substance or composition to be received therein. The solid filler material may be fumed silica Aerosil 200, commercially available product obtainable from Degussa Africa (Pty) Limited.

The patch of the present invention further preferably provides a passage through the distal layer, and, where present, the adhesive layer and impermeable high density polypropylene layer, and through which passage the substance or composition to be administered to the patient may in use be introduced into the cavity of the patch after the latter had been placed on the patient. The passage is preferably in part constituted by a self-reclosing nipple or port formation integrally moulded with the distal layer of the patch construct, or part thereof, to present an access opening on the outer surface of the patch into which the spout-like needle mounting of a conventional syringe may in use be received to introduce the substance or composition to be administered into the cavity via the passage. In the preferred application of the invention the composition to be administered to the patient with the aid of the patch of the invention is contained in the required quantity and strength in a sealed tube as an ointment or cream of the desired consistency. The tube is preferably provided with a spout which is then in use inserted into the self-reclosing nipple and the content thereof is squeezed out into the cavity.

The patch construct may further include a self-adhesive mounting layer having a surface presenting a skin adhesive which adhesive layer is typically during pre-use storage of the product covered by a peelable cover layer, which cover layer also overlies, and hence seals off, the proximal layer of the patch construct until it is exposed by peeling off the cover layer. The mounting layer may be in the form of a commercially available product conventionally used in the production of skin plasters and sold under the name Flexifix®.

### EXAMPLE OF THE INVENTION

An example of the present invention will now be described merely to illustrate the invention and without thereby limiting the scope of the invention to the exemplary embodiment.

In the accompanying drawings

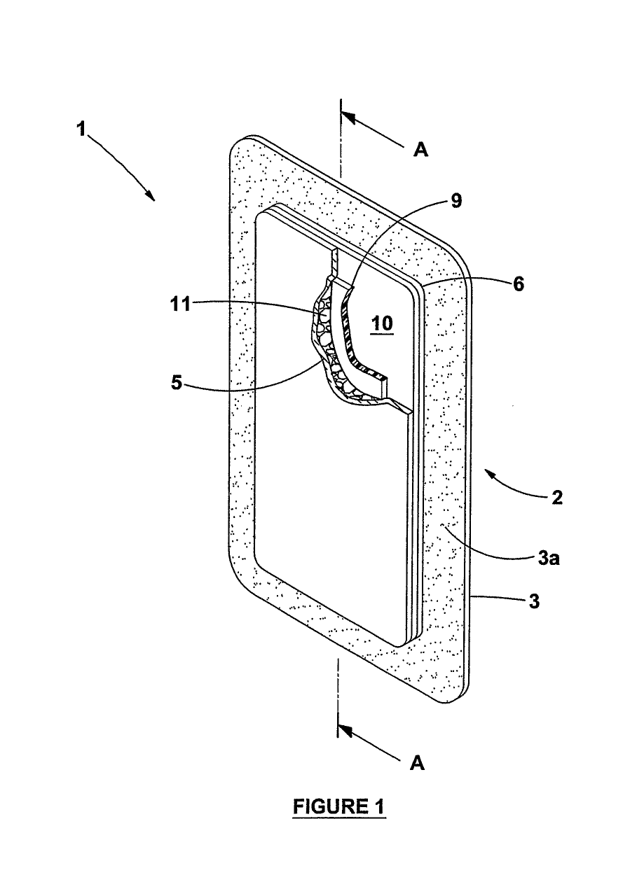

FIG. 1 is a schematic partly broken away perspective view of a patch according to the invention viewed from the side of the proximal layer thereof;

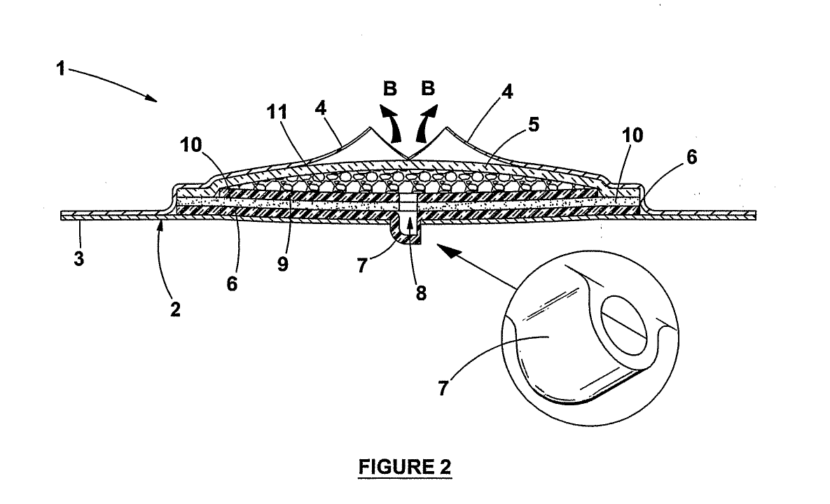

FIG. 2 is a cross-sectional elevation of the patch of FIG. 1 along the line A-A-, and showing a detailed perspective view of the port formation forming part of it.

It is emphasized that the drawings are not to scale, and are of a schematic nature. The dimensions of some parts of the patch are greatly exaggerated for illustrative purposes.

Reference will now be made to the accompanying drawings in which the same reference numerals are used to indicate the same elements. A trans-dermal patch 1 is shown to comprise a mounting sheet 2 comprising a backing layer 3 and a peelable cover 4, which cover is not shown in FIG. 1 but only in FIG. 2 where arrows B show the typical directions in which the peelable cover may be removed to expose the proximal layer 5 of the patch. The skin adhesive coated surface of the backing layer 3 may, as pointed out above, be in the form of the commercially available product conventionally used in the production of skin plasters sold under the name Flexifix®.

Onto the adhesive coated backing layer 3 is mounted a press vulcanized or injection moulded silicone distal layer 6 of the patch according to the invention which distal layer 6 is moulded in the form of a sheet having a thickness of 0.5 mm and having in the centre thereof an access port 7 in the form of a nipple having a terminal membrane adapted to be slit to allow the needle mounting spout of a conventional syringe or the spout of a tube containing the composition to be introduced into the passage 8 extending from the slit to the inner surface of the distal layer. The distal layer is preferably press or injection moulded from a silicone composition marketed by Wacker-Chemie GmbH of Germany under the trade name Elastosil® R401/70 to have the required properties. It may be suitably coloured by the addition of a compatible dye.

A layer of DMF impervious membrane in the form of high density polypropylene layer 9 is applied to the inner surface of the distal layer by interposing between the silicone distal layer 6 and the impervious high density polypropylene layer 9 a suitable silicone adhesive transfer tape 10 which is sold under the trade name RBB577B by PPI Adhesive Products of Waterford in Ireland.

A proximal layer of silicone produced from Elastosil® R401/70 to a thickness of 0.25 mm, which is indicated at 5 is applied over the high density polypropylene barrier layer 9 and secured in place by means of the adhesive on the peripheral surface of the silicone adhesive tape indicated by reference number 10, so as to create between the proximal layer 5 and the composite distal layer composed of the silicone layer 6 and the adhesively attached high density polypropylene layer 9 attached thereto a cavity 11 which is in communication with the port 7 via apertures provided in the high density polypropylene layer 9 and the adhesive layer 10. The construct is completed by the peelable cover indicated at 4 in FIG. 2 [but not shown in FIG. 1] whereby the product as a whole is sealed during storage.

The cavity 11 defined between the proximal layer 5 and the composite distal layer 6 of the patch may for some applications be provided with a charge of fumed silica Aerosil which is capable of acting as a carrier for the substance to be administered, or may be left void if the patch is to be used to administer an active substance in a paste, which may itself contain fumed silica Aerosil as a carrier for DMF containing pharmaceutical preparation which is to be introduced into the cavity for administration to a patient.

In use, the peelable layer 4 is stripped from the construct to reveal the skin adhesive presented by the peripheral zone of the backing layer 3 as shown at 3a and the patch is then applied to the human or animal body which is to receive treatment.

Once placed and adhered in position on the body of the patient, e.g. on the chest, upper arm, on a shoulder blade or on the thigh of the patient, a required quantity of the pharmaceutical preparation to be administered is introduced by a syringe or tube into the cavity by rupturing the self-closing split in the nipple formation 7 of the patch which is best seen in the detailed perspective bubble in FIG. 2.

The application of a DMF containing pharmaceutical preparation by means of the patch according to the invention has been tested on patients and compared to the experience gained with similar patches of a conventional construction. Whereas patients using conventional patches with the composition in issue experienced extensive irritation and skin burns in the form of pruritic erythema, such irritation and burning sensation was substantially reduced or completed absent when the plaster of the present invention was used.

Many variations of the invention are possible without thereby departing from the spirit of the invention as disclosed herein. Thus, the patch may be produced in any desired shape and may, for example, be circular in shape. Also, it is not essential for the patch to be provided with a nipple formation as shown in the drawings. The patch may thus have a fully enclosed cavity into which a breakable sachet containing the substance to be administered by means of the patch is enclosed during production of the patch. In use the sachet may be broken without damage to the layers of the patch itself immediately before the patch is applied to the skin.

1. A patch device adapted for use in the transdermal administration to a patient of a composition including, or consisting of, dimethylformamide (DMF), the patch consisting of a layered construct adapted to be adhered to the skin of a patient and defining a depot cavity for the composition to be administered between a proximal layer and a distal layer thereof, which proximal layer is adapted in use to be located in intimate contact with the skin of the patient and which distal layer is in use disposed on the outer side thereof, the distal layer being characterized in that it is flexible and made of an elastomeric silicone material and is substantially impervious to the composition to be administered, and the proximal layer being characterized in that it is flexible and made of an elastomeric silicone material and is partially permeable to that composition, so that in use the composition may be disposed in the cavity and permeate from there through the proximal layer to be absorbed through the skin into the body of the patient, the proximal and distal layers being bonded to portions of each other by means of silicone adhesive or by vulcanizing, the proximal layer being further characterized in that its permeability to the irritating substance or component of the composition to be administered to the human or animal is less than the permeability of the human or animal skin, as the case may be, to such irritating substance or component.

2. The patch of claim 1 wherein the chemical composition of the proximal and distal layers are the same, and the respective permeability and impervious characteristics of the layers are achieved by virtue of the thicknesses of the respective layers.

3. The patch of claim 1 wherein the chemical composition of the proximal and distal layers are the same and the distal layer is modified by the application thereto of a thin impervious layer to render a composite layer which is impervious to the components of the composition to be applied by means of the patch.

4. A patch according to claim 1 suitable for use in the administration of DMF to a patient and characterized in that the proximal layer of the patch has a permeability to dimethylformamide (DMF) such that DMF which is, in use, located in the cavity between the layers will be released through the proximal layer at a rate below the rate at which it is absorbed through the skin of the patient to which the patch is in use applied, thereby substantially preventing building-up of DMF in direct contact with the skin of the patient.

5. The patch of claim 1 which is adapted to be adhered to the skin of the patient by having a peripheral edge zone of the proximal layer which is provided with a pharmaceutically acceptable adhesive layer, which adhesive layer is covered by means of a conventional peel-off cover sheet during storage.l

6. The patch of claim 4 in which the layers of a patch intended to be used for the administration of dimethylformamide as such, or of a pharmaceutical preparation containing dimethylformamide as a penetration enhancing agent, are of the same chemical composition and are made from vulcanizates of silicone.

7. The patch of claim 6 wherein the composition of the layer intended for use as a proximal layer in a patch for use in the administration of dimethylformamide (DMF) as such, or of a pharmaceutical preparation containing dimethylformamide as a penetration enhancing agent, is produced to have a permeability to dimethylformamide of not more than 9 mg DMF/cm²/hour.

8. The patch of claim 1 in which the cavity defined between the proximal and distal layers of the patch construct is preferably in use filled with a solid filler material to serve as a carrier for the pharmaceutically active substance or composition to be received therein.

9. The patch of claim 1 characterized in that it provides a passage through the distal layer or layers, through which passage the substance or composition to be administered to the patient may in use be introduced into the cavity of the patch after the patch has been placed on the patient.

10. The patch of claim 9 wherein the passage is in part constituted by a self-reclosing nipple or port formation integrally moulded with the distal layer of the patch construct, or part thereof, to present an access opening on the outer surface of the patch into which the spout-like needle mounting of a conventional syringe may in use be received to introduce the substance or composition to be administered into the cavity via the passage.

11. The patch of claim 1 wherein the construct further includes a self-adhesive mounting layer having a surface presenting a skin adhesive which adhesive layer is typically during pre-use storage of the product covered by a peelable cover layer, which cover layer also overlies, and hence seals off, the proximal layer of the patch construct until it is exposed by peeling off the cover layer.
